(19) United States
(12) Patent Application Publication
(10) Pub. No.: US 2018/0161164 A1
(43) Pub. Date: Jun. 14, 2018

(54) POSTERIORLY STABILIZED TOTAL KNEE JOINT PROSTHESIS

(71) Applicant: Shanghai Xinjian Medical Co., LTD., Shanghai City (CN)

(72) Inventor: FEI LIU, Shanghai City (CN)

(21) Appl. No.: 15/390,493

(22) Filed: Dec. 24, 2016

(30) Foreign Application Priority Data
Dec. 12, 2016 (CN) 201611137209.8

(51) Int. Cl. A61F 2/38

(52) U.S. Cl. CPC A61F 2/389; A61F 2/3859

(57) ABSTRACT

A posteriorly stabilized total knee joint prosthesis comprises a femoral component and a tibial component articulating with each other. The tibial component includes a stabilizing post with a spine articular surface at the posterior thereof. The femoral component has a convex cam follower interacting with the stabilizing post. The convex cam follower configured with a non-cylindrical geometries includes four spine contacting portions gradually extending posteriorly further in order. The last three spine contacting portions define a cam articular surface bearing on the spine articular surface of the post. The cam articular surface has decreasing curvature radiuses along the posterior extension of the cam follower, and the spine articular surface has a concave cam surface with a curvature radius slightly bigger than that of the last spine contacting portion along the extension of the cam follower.

### BACKGROUND OF THE INVENTION

### Field of the Invention

The present invention relates to knee joint prostheses for replacing the articular surfaces of a diseased or injured human knee, more particularly to an implantable knee joint prosthesis which replaces all surfaces of the femur and tibia which engage each other at the knee joint and stabilizes joint function in leg flexion.

### Description of the Prior Art

Disease and trauma affecting the articular surfaces of the knee joint are commonly effectively treated by surgically replacing the articulating ends of the femur and tibia with prosthetic femoral and tibial implants, referred to as total knee replacements (TKR). These implants are made of materials that exhibit a low coefficient of friction as they articulate against one another so as to restore normal knee function. Modern TKR's are tricompartmental designs. The patella-femoral joint and the lateral and medial inferior tibial-femoral joints replace three separate articulating surfaces within the knee joint. These implants are designed to articulate from a position of slight hyperextension to approximately 115 to 130 degrees of flexion.

Such a tricompartmental design can meet the needs of most TKR patients even though the healthy human knee is capable of a range of motion (ROM) approaching 170 degrees. However, there are some TKR patients who have particular need to obtain very high flexion in their knee joint, usually as a result of cultural considerations. For many in the orient, and for some in the west, a TKR which permits a patient to achieve a ROM in excess of 150 degrees is desirable to allow deep kneeling, squatting, and sitting on the floor with the legs tucked underneath.

### SUMMARY OF THE INVENTION

In accordance with the present invention, a posteriorly stabilized total knee joint prosthesis is provided to retain a stable interaction between the cam flower of the femoral component and the post of the tibial component under the condition of high curvature movement.

In order to achieve the object set forth, a posteriorly stabilized total knee prosthesis comprising a femoral component and a tibial component articulating with the femoral component; the femoral component including arcuate medial and lateral condylar portions joined together to form a patellar portion and a recess surrounded by the condylar portions and the patellar portion together; the tibial component including a disc-like plateau portion with a pair of spaced-apart, oblong concavities for respectively receiving the medial and lateral condylar portions, and a stabilizing post received in the recess of the femoral component; the stabilizing post extending superiorly from the plateau portion and defining a spine articular surface at the posterior thereof; wherein the femoral component has a convex cam follower interacting with the stabilizing post of the tibial component, and wherein the convex cam follower configured with a non-cylindrical geometries includes four spine contacting portions gradually extending posteriorly further in order, and wherein the last three spine contacting portions together define a cam articular surface bearing on the spine articular surface of the stabilizing post, and wherein the cam articular surface has decreasing curvature radiuses along the posterior extension of the cam follower, and wherein the spine articular surface has a concave cam surface with a curvature radius slightly bigger than that of the last spine contacting portion along the extension of the cam follower.

Other objects, advantages and novel features of the present invention will become more apparent from the following detailed description when taken in conjunction with the accompanying drawings, in which:

### BRIEF DESCRIPTION OF THE DRAWINGS

FIG. 1 is an exploded, perspective view of a posteriorly stabilized total knee joint prosthesis in accordance with the preferred embodiment of the present invention;

FIG. 2 is a side view of the posteriorly stabilized total knee joint prosthesis with the femoral component assembled on the tibial component shown in FIG. 1;

FIG. 3 is a side view of the femoral component of the posteriorly stabilized total knee joint prosthesis shown in FIG. 1;

FIG. 4 is a cross-sectional view of the femoral component shown in FIG. 3;

FIG. 5 is a perspective view of the tibial component shown in FIG. 1; and

FIGS. 6-9 are side plane views of the total knee joint prosthesis of the present invention, showing the femoral component articulating with the tibial component between 60 degrees and 145 degrees of flexion.

### DESCRIPTION OF PREFERRED EMBODIMENTS OF THE INVENTION

Reference is now made to the drawings to describe the invention in detail.

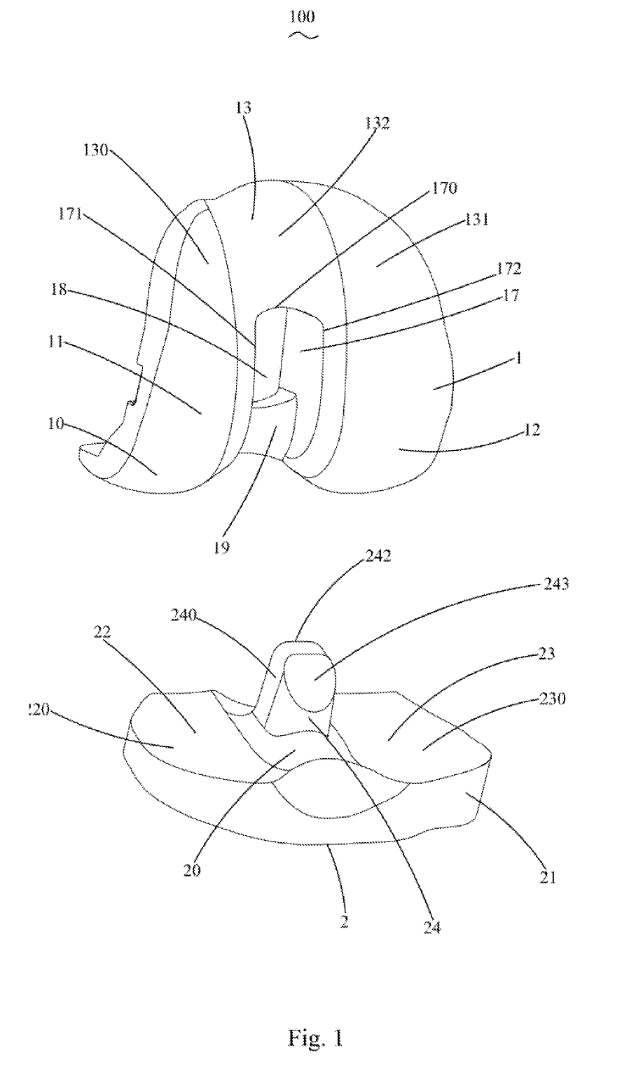

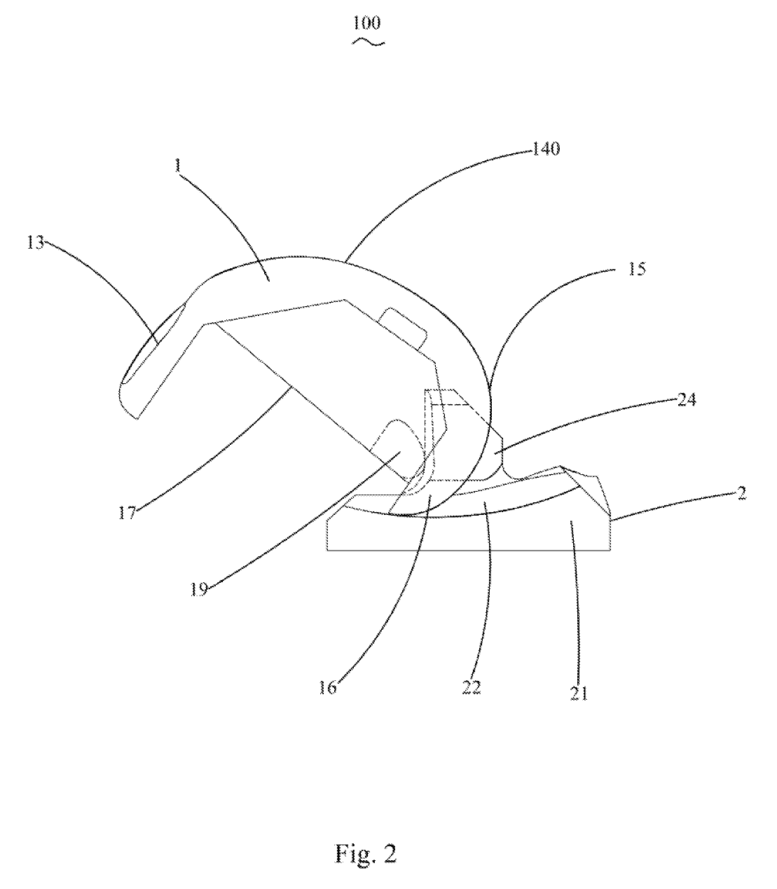

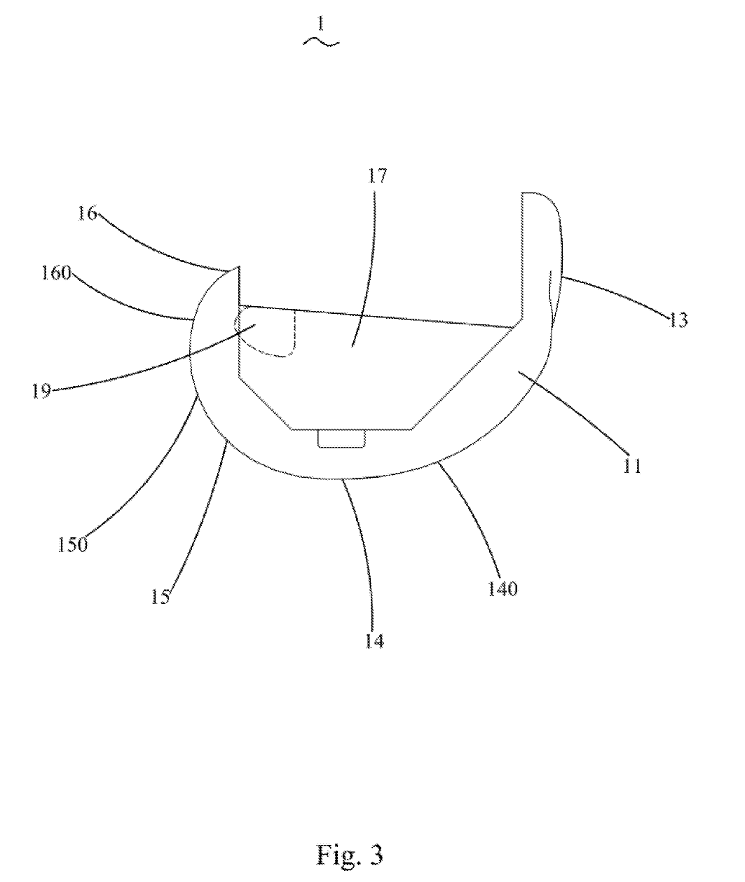

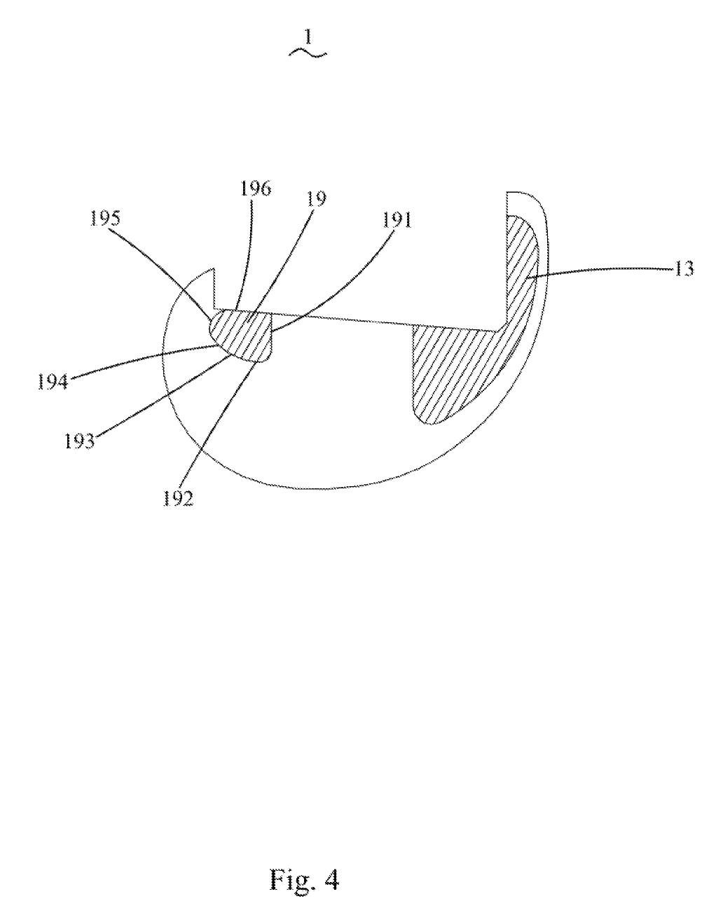

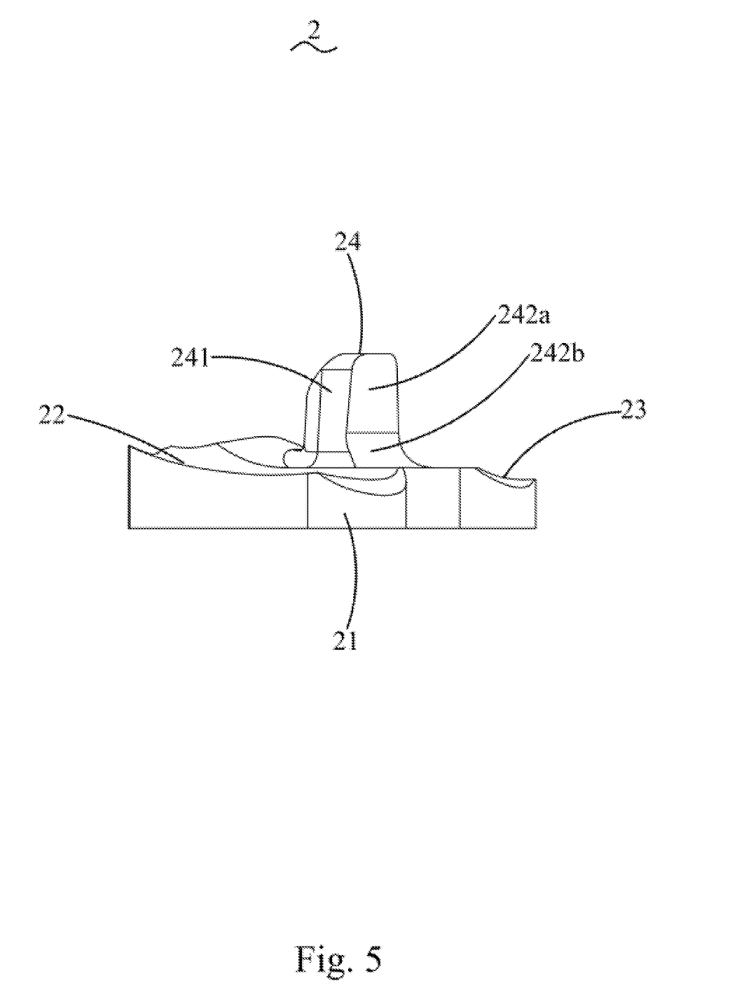

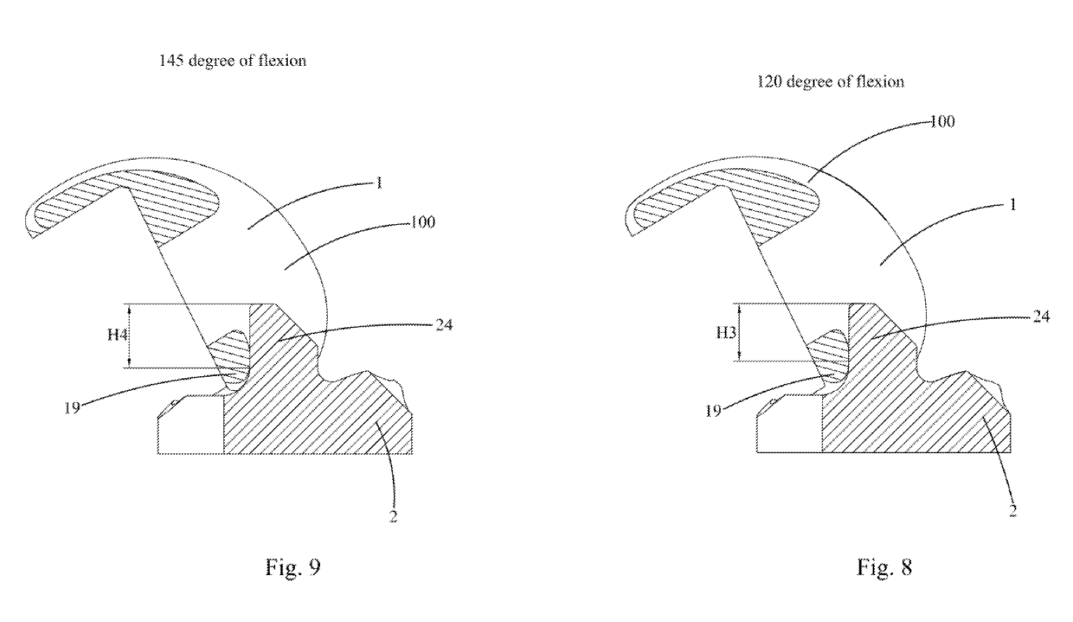

As illustrated in FIGS. 1-9, a preferred embodiment of this invention provides a posteriorly stabilized total knee prosthesis 100 having an improved stability under the condition of high curvature movement. The total knee prosthesis 100 replaces all surfaces of the femur and tibia engaging with each other at the knee joint and comprises a femoral component 1 and a tibial component 2 engaging with each other.

Referring to FIGS. 1-4, the one-piece femoral component 1 includes a pair of laterally spaced-apart condylar portions respectively named as a medial condylar portion 11 and a lateral condylar portion 12. Each of the medial and lateral condylar portions 11, 12 is smoothly convexly curved in lateral profile generally to match the curvature of an anatomical femoral condyle and is laterally convexly curved entirely along an anterior-posterior extent thereof. The medial and lateral condylar portions 11, 12 both are arcuate shaped and joined together at the anterior aspects thereof to form a patellar portion 13.

Each of the medial and lateral condylar portions 11, 12 includes a distal condylar portion 14 extending distally from the patellar portion 13 to form a distal articular surface 140, a posterior condylar portion 15 extending posteriorly from the distal condylar portion 14 to form a posterior articular surface 150 and a superior condylar portion 16 extending superiorly from the posterior condylar portion 15. The superior condylar portion 16 extends to form a superior surface 160 back toward the patellar portion 13. The patellar portion 13, the distal condylar portion 14, the posterior condylar portion 15 and the superior condylar portion 16 define a smooth articular surface 10 extending around the exterior of the femoral component 1. The smooth articular surface 10 is configured with the distal, posterior and superior articular surfaces 140, 150, 160.

The patellar portion 13 is configured with a laterally convex and inferior-superiorly concave structure and has two convexly curved lateral portion 130, 131 and a concave medial portion 132 smoothly merging with the convexly curved lateral portions 130, 131. Two convexly curved lateral portion 130,131 respectively merge smoothly with the anterior aspects of the medial and lateral condylar portions 11, 12. The femoral component 1 defines a box-like intercondylar portion 17 at the interior thereof and a recess 18 surrounded by the intercondylar portion 17.

The intercondylar portion 17 connects the medial and lateral condylar portions 11, 12 together with the patellar portion 13. The intercondylar portion 17 has an anterior roof 170 intersecting the concave medial portion 132 of the patellar portion 13. The concave medial portion 132 of the patellar portion 13 intersects the anterior roof 170 at the inferior extremity thereof. A pair of laterally spaced-apart sidewalls 171, 172 of the intercondylar portion 17 respectively join the edges of the anterior roof 170 to the internal edges of the medial and lateral condylar portion 11, 12. The recess 18 defined by the intercondylar portion 17 allows fluids and tissue more readily to enter and grow thereinto for better integration of the posteriorly stabilized total knee prosthesis 100 with anatomical structures and systems.

The femoral component 1 engaging with the tibial component 2 includes a convex cam follower 19 respectively connecting the medial and lateral condylar portions 11, 12. The cam follower 19 is located at the posterior-superior extremities of the medial and lateral condylar portions 11, 12. The recess 18 is surrounded by the anterior roof 170 of the intercondylar portion 17, laterally spaced-apart sidewalls 171, 172 of the intercondylar portion 17 and the convex cam follower 19. The cam follower 19 configured with non-cylindrical geometries includes a relatively flat portion 191, a first spine contacting portion 192 having a first curvature radius, a second spine contacting portion 193 having a second curvature radius, a third spine contacting portion 194 having a third curvature radius and a fourth spine contacting portion 195 having a fourth curvature radius. The first curvature radius of the first spine contacting portion 192 is smaller than that of the second spine contacting portion 193. The third curvature radius of the third spine contacting portion 194 is smaller than that of the second spine contacting portion 193. The fourth curvature radius of the fourth spine contacting portion 195 is smaller than that of the third spine contacting portion 194.

The first spine contacting portion 192 is an arc of the circle defined by the first radius. The second spine contacting portion 193 is an arc of the circle defined by the second radius and extends further posteriorly than the perimeter of the circle defined by the first radius. The third spine contacting portions 194 is an arc of the circle defined by the third radius and extends further posteriorly than the perimeter of the circle defined by the second radius. The fourth spine contacting portion 195 extending toward the top 196 of the cam follower 19 is an arc of the circle defined by the fourth radius and forms the posterior most cam surface and the end of the cam articular surface. Four spine contacting portions 192, 193, 194, 195 form an ovoid articular surface 190 engaging the corresponding portion of the tibial component 2 in high flexion. The last three spine contacting portions 193, 194, 195 forms a cam articular surface with decreasing curvature radiuses along the posterior extension of the cam follower 19. The top portion 196 of the cam follower 19 completes the cam profile.

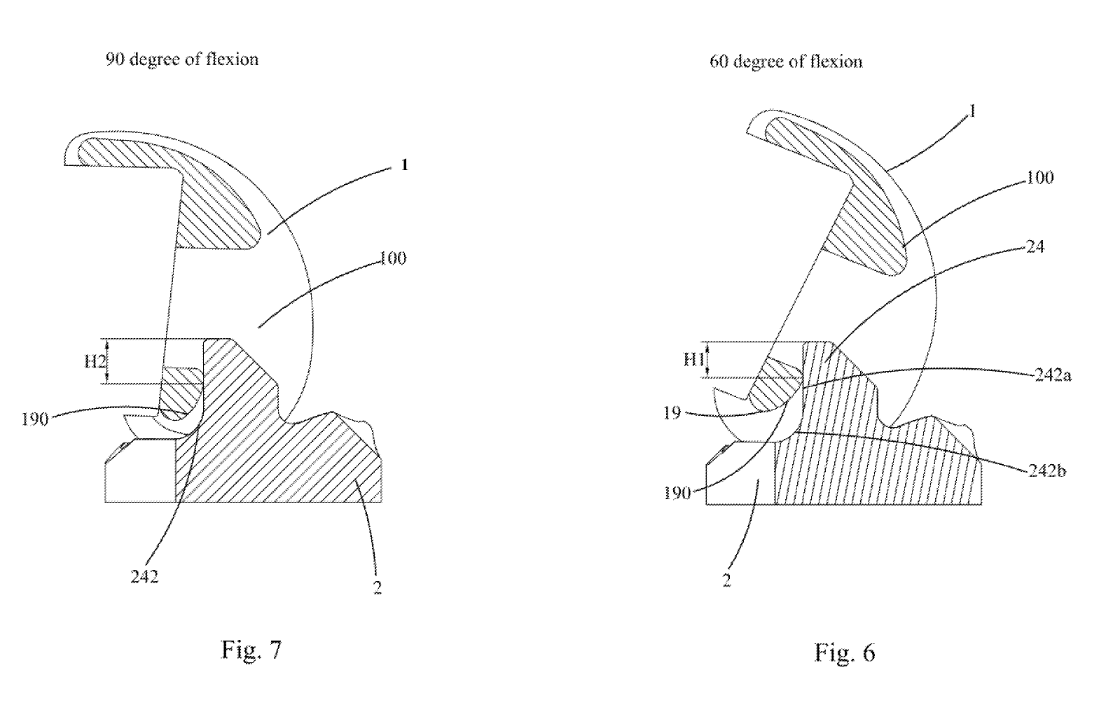

Referring to FIGS. 1, 2 and 5, the tibial component 2 engaging with the femoral component 1 is symmetrical about a vertical anterior-posterior center plane and comprises an oblong, rounded, disc-like plateau portion 21 having a generally flat upper surface 20 sloping down from front to back. A pair of laterally spaced-apart, oblong concavities 22, 23 are formed through downwardly depressing the plateau portion 21 for respectively receiving the corresponding condylar portions 11, 12. The laterally spaced-apart, oblong concavities 22, 23 each defines an articular surface 220, 230 corresponding to the smooth articular surface 10 of the femoral component 1. A stabilizing post 24 extends superiorly from the plateau portion 21 and is located between the concavities 22, 23. The stabilizing post 24 received in the recess 18 of the femoral component 1 interacts the convex cam follower 19 of the femoral component 1 to create a center for rotation of the femoral component 1 relative to the tibial component 2. A post/cam contacting point is formed during the cam follower 19 of the femoral component 1 bears on the post 24 of the tibial component 2. The distance from the post/cam contacting point to the top of the post 24 is called the jump height and gradually increased as enlarging the degree of flexion shown in FIGS. 6-9, so as to prevent anterior subluxation of the femoral component 1 relative to the tibial component 2.

The stabilizing post 24 structured with a generally trapezoidal shape in lateral profile has flat, parallel lateral surfaces 240 and 241, a posterior surface 242 and an anterior surface 243. The lateral surfaces 240 and 241 are in sufficient clearance from the lateral sidewalls 171, 172 of the intercondylar portion 17 to allow normal lateral angulation and rotation of the total knee prosthesis 100. The anterior surface 243 slopes anteriorly and superiorly at an acute included angle to a nominal reference plane perpendicular to the nominal axis of the extended leg. The posterior surface 242 is the articular surface of the stabilizing post 24 and is bore the articular surface 190 of the convex cam follower 19 in leg flexion.

The posterior surface 242 has a flat surface 242a at the superior thereof and a concave cam surface 242b at the inferior thereof. The flat surface 242a configured as a vertical surface along the extension of the post 24 is bore the articular surface 190 of the convex cam follower 19 during the femoral component 1 articulating with the tibial component 2 between 45 degrees and 60 degrees of flexion. The concave cam surface 242b has a curvature radius slightly bigger than that of the fourth spine contacting portion 195 of the convex cam follower 19 and is bore the articular surface 190 of the convex cam follower 19 during the femoral component 1 articulating with the tibial component 2 between 120 degrees and 150 degrees of flexion.

Furthermore, although the present invention has been described with reference to particular embodiments, it is not to be construed as being limited thereto. Various alterations and modifications can be made to the embodiments without in any way departing from the scope or spirit of the present invention as defined in the appended claims.

What is claimed is:

1. A posteriorly stabilized total knee joint prosthesis comprising:
a femoral component including arcuate medial and lateral condylar portions joined together to form a patellar portion and a recess surrounded by the condylar portions and the patellar portion together; and
a tibial component articulating with the femoral component and including a disc-like plateau portion with a pair of spaced-apart, oblong concavities for respectively receiving the medial and lateral condylar portions, and a stabilizing post received in the recess of the femoral component, the stabilizing post extending superiorly from the plateau portion and defining a spine articular surface at the posterior thereof;
wherein the femoral component has a convex cam follower interacting with the stabilizing post of the tibial component, and wherein the convex cam follower configured with a non-cylindrical geometries includes four spine contacting portions gradually extending posteriorly further in order, and wherein the last three spine contacting portions together define a cam articular surface bearing on the spine articular surface of the stabilizing post, and wherein the cam articular surface has decreasing curvature radiuses along the posterior extension of the cam follower, and wherein the spine articular surface has a concave cam surface with a curvature radius slightly bigger than that of the last spine contacting portion along the extension of the cam follower.

2. The posteriorly stabilized total knee joint prosthesis of claim 1, wherein the four spine contacting portions are all arcs of different circles defined by different curvature radiuses, and wherein the curvature radiuses of the first two spine contacting portions become bigger.

3. The posteriorly stabilized total knee joint prosthesis of claim 1, wherein the convex cam follower has an ovoid articular surface defined by the four spine contacting portions, and wherein the spine articular surface includes a vertically flat surface at the superior thereof, and the vertically flat surface is bore the ovoid articular surface of the cam follower in low flexion, and wherein the concave cam surface is bore the ovoid articular surface of the cam follower in high flexion.

4. The posteriorly stabilized total knee joint prosthesis of claim 1, wherein the last spine contacting portion extends toward the top of the cam follower and forms the posterior most cam surface and the end of the cam articular surface.

5. The posteriorly stabilized total knee joint prosthesis of claim 1, wherein the femoral component includes a box-like intercondylar portion joining the medial and lateral portions, and wherein the recess extends inferiorly through the intercondylar portion toward the tibial component.

6. The posteriorly stabilized total knee joint prosthesis of claim 5, wherein the convex cam follower forms the posterior wall of the intercondylar portion, and wherein the intercondylar portion includes an anterior roof intersecting the concave medial portion of the patellar portion.

7. The posteriorly stabilized total knee joint prosthesis of claim 6, wherein the intercondylar portion has a pair of laterally spaced-apart sidewalls respectively joining the edges of the anterior roof to the internal edges of the medial and lateral condylar portion.

8. The posteriorly stabilized total knee joint prosthesis of claim 6, wherein the patella portion configured with a laterally convex and inferior-superiorly concave structure has two convexly curved lateral portion respectively merging smoothly with the anterior aspects of the medial and lateral condylar portions.

9. The posteriorly stabilized total knee joint prosthesis of claim 8, wherein the concave medial portion of the patella portion smoothly merges with the convexly curved lateral portions.

10. The posteriorly stabilized total knee joint prosthesis of claim 1, wherein each of the medial and lateral condylar portions has an arcuate shape, and wherein the patella portion is formed at the anterior aspects of the medial and lateral condylar portions.

11. The posteriorly stabilized total knee joint prosthesis of claim 1, wherein each of the medial and lateral condylar portions is smoothly convexly curved in lateral profile, and wherein the recess is defined by the intercondylar portion and the cam follower together.

12. The posteriorly stabilized total knee joint prosthesis of claim 1, wherein each of the medial and lateral condylar portions includes a distal condylar portion extending distally from the patellar portion to form a distal articular surface, a posterior condylar portion extending posteriorly from the distal condylar portion to form a posterior articular surface and a superior condylar portion extending superiorly from the posterior condylar portion, and wherein the cam follower respectively connects the superior condylar portions.

13. The posteriorly stabilized total knee joint prosthesis of claim 12, wherein a smooth articular surface is configured with the distal, posterior and superior articular surfaces and extends around the exterior of the femoral component.

14. The posteriorly stabilized total knee joint prosthesis of claim 1, wherein each of the oblong concavities is formed by downwardly depressing the plateau portion and defines an articular surface corresponding to the smooth articular surface of the femoral component.

15. The posteriorly stabilized total knee joint prosthesis of claim 1, wherein the stabilizing post is located between the oblong concavities.

16. The posteriorly stabilized total knee joint prosthesis of claim 1, wherein the total knee joint prosthesis has a post/cam contacting point formed during the cam follower of the femoral component bears on the post of the tibial component, and wherein the distance from the post/cam contacting point to the top of the post is gradually increased as the degree of flexion becoming bigger.

17. The posteriorly stabilized total knee joint prosthesis of claim 5, wherein the stabilizing post defines flat, parallel lateral surfaces each having a sufficient clearance from the lateral sidewalls of the intercondylar portion.

18. The posteriorly stabilized total knee joint prosthesis of claim 17, wherein the stabilizing post has an anterior surface sloping anteriorly and superiorly at an acute included angle to a nominal reference plane perpendicular to the nominal axis of the extended leg.
